(19) United States
(12) Patent Application Publication
(10) Pub. No.: US 2025/0099713 A1
(43) Pub. Date: Mar. 27, 2025

(54) COIL CATHETER METHOD OF MANUFACTURE

(71) Applicant: Blue Halo BioMedical, LLC, Winter Park, FL (US)

(72) Inventors: Gaines Hammond, JR., Greenville, SC (US); Byron Hodge, JR., Lakeland, FL (US); Gary J. Mishkin, Potomac, MD (US); Anthony R. Ruben, Winter Park, FL (US)

(73) Assignee: Blue Halo BioMedical, LLC, Winter Park, FL (US)

(21) Appl. No.: 18/971,706

(22) Filed: Dec. 6, 2024

Related U.S. Application Data
(63) Continuation of application No. 17/863,795, filed on Jul. 13, 2022, now Pat. No. 12,194,246.
Application No. 17/863,795 is a continuation of application No. 17/850,389, filed on Jun. 27, 2022.
Application No. 17/850,389 is a continuation-in-part of application No. 17/012,920, filed on Sep. 4, 2020, now abandoned.
(60) Provisional application No. 62/896,724, filed on Sep. 6, 2019.

(51) Int. Cl. A61M 25/00; A61F 2/04; A61M 27/00; B29C 33/02; B29D 23/00

(52) U.S. Cl. CPC A61M 25/0009; A61F 2/04; A61M 27/008; B29C 33/02; B29D 23/00; A61F 2002/047; A61F 2002/048; A61F 2210/0014; A61F 2220/0008; A61F 2220/0075; A61F 2230/0091; A61F 2240/001; A61F 2240/004; A61M 2210/1089

(57) ABSTRACT

A catheter device and manufacturing process for manufacturing the catheter device, wherein the catheter device has a halo-shaped coiled portion extending away from a perpendicular stem portion through a swan neck portion. Eyelets on the halo coil portion, and swan neck portion facilitate flow out of the bladder through the catheter device vertical to the catheter, rather than perpendicularly as is the case with existing catheters. The catheter device is formed by using a straight catheter tube, heating and cooling it within a formed mold to have the halo coil and swan neck, such that it can be straightened using a pusher and stylet, inserted into the body while straightened, and thereafter return to its coiled shape when the stylet is removed.

### CROSS-REFERENCE TO RELATED APPLICATIONS

This application is a continuation of Nonprovisional patent application Ser. No. 17/863,795, filed on Jul. 13, 2022, and entitled “COIL CATHETER METHOD OF MANUFACTURE”, which is a continuation of U.S. Nonprovisional patent application Ser. No. 17/850,389, filed on Jun. 27, 2022, and entitled “COIL CATHETER, METHOD OF USE, AND METHOD OF MANUFACTURE”, which is a continuation-in-part of U.S. Nonprovisional patent application Ser. No. 17/012,920, filed on Sep. 4, 2020, and entitled “COIL CATHETER, METHOD OF USE, AND METHOD OF MANUFACTURE”, which claims the benefit of priority of U.S. Provisional Application Ser. No. 62/896,724, filed on Sep. 6, 2019, and entitled “HALO COIL CATHETER AND METHOD OF USE”. The entirety of U.S. Nonprovisional patent application Ser. No. 17/863,795, U.S. Nonprovisional patent application Ser. No. 17/850,389, U.S. Nonprovisional patent application Ser. No. 17/012,920, and U.S. Provisional Patent Application Ser. 62/896,724 are incorporated herein by reference.

### FIELD OF THE INVENTION

The present invention relates generally to a coil catheter, method for use thereof, and method of manufacture thereof. More specifically, the present invention relates to a catheter having a retention coil member for repositioning, validation of placement, and removal, method of use, and method of manufacture thereof.

### BACKGROUND

Urinary retention in males generally implies that urine is produced normally but is retained within the bladder due to primary detrusor dysfunction or an obstruction at or distal to the bladder neck. Prostatic obstruction may cause an increased resistance for the passage of urine with subsequent increased pressure on the bladder musculature resulting in two successive phases: 1) compensation, where bladder emptying may still take place, and 2) decompensation, characterized by increasing amounts of residual urine and ultimately urine retention. Bladder outlet obstruction producing urinary retention may result from, including but not limited to, benign prostatic hypertrophy, prostate cancer, or any acute enlargement of the prostate including but not limited to acute prostatitis, post focal procedures on the prostate, radiation, cryotherapy, or instrumentation. Urinary retention occurs primarily in males, with benign prostatic hypertrophy being the most common cause in men over the age of 50 years. It has been estimated that one in four men in the United States will have been treated for symptomatic benign prostatic hypertrophy by the time they reach 80 years of age.

The prior art contains several examples, including devices which are disclosed in U.S. Pat. No. 7,044,980, incorporated herein by reference. Relevant, but distinguishable, prior art also includes U.S. Pat. No. 4,738,667 which teaches a halo-style catheter which presents additional issues that would need to be solved. For example, existing concepts and systems require external draining and a sheath requiring a bulbous end which was designed, at the time, to avoid stiff guidewires. New guidewires for catheter placement have reduced this stiffness, and are superior to such sheathes as taught in the prior art. What is desirable is a tapered end with a guidewire for ease of placement.

Material choices in existing systems can also be problematic. For example, Dacron (a cloth-like material) is taught by the prior art for use in coating a catheter. This material can become highly lithogenic when exposed to urine, so it would be ideal to use an alternative material or method. Another issue taught in the prior art is a sheath which becomes a permanent part of the system required to straighten the catheter for insertion and is further used to uncoil and retract the catheter from the bladder. It would be desirable not to require a permanently placed sheath to remove additional obstacles during the operation of a catheter.

It is also taught through prior systems to transverse the external sphincter of the bladder. This results in incontinence, even though the bladder may be draining through the catheter, because the sphincter is required to be kept open due to the presence of existing catheters. It is desirable to shorten the upper arm of such a catheter such that it sits above the urinary sphincter rather than retaining that sphincter in an open orientation.

Management for urinary retention related to prostate obstruction involves bladder drainage generally accomplished by placement of a transurethral, suprapubic catheter or intermittent self-catheterization. This creates a passageway between the bladder and the exterior of the body that allows a flow of urine to the outside.

Heretofore there has not been available a system or method for a catheter with the advantages and features of the present invention.

### SUMMARY OF THE DISCLOSURE

The invention involves facilitating drainage and, more specifically, but not by way of limitation, facilitating fluid drainage from the bladder and through the urethra of a patient experiencing either acute or chronic urinary obstruction.

The retaining member for the device is a coil device which has a straight non-coiled iteration for placement. Once placed into the bladder, a stylet may be removed, which allows for a coiled default state to be achieved. A pusher segment may be connected during placement and may be utilized for a period of time to collect urine and then removed, allowing the device to have only a thread (which may be constructed using monofilament suture-type material) connected externally. A coiled tubular portion abutted to the pusher segment may allow for passage and collection of urine. Upon disconnection from the pusher segment, the coil catheter may allow for the passage of urine without an external tubular structure or external collection device. The converted iteration may be from a long external catheter to a short coil device without a tubular extension through the sphincter or distal urethra.

In general, and in one aspect, the invention relates to a catheter system, which embodies the ability to control a coil tip catheter within a bladder or body cavity by an attached thread traversing through a urethra to the exterior. The coil catheter may include a body member and a coil retaining member. The body member may include a distal terminating end, a proximal end portion, and a lumen extending within the body member to allow fluid drainage through the body member as well as around the tubular segment. Eyelets may be placed in the device at but not limited to a swan neck portion and/or a proximal coil tip. A guide wire opening at the tip may allow for use of a guide wire to facilitate delivery of the device due to urethral or prostate anatomical challenges.

The directional terms proximal and distal may require a point of reference. In this application, the point of reference in determining a direction is from the perspective of a patient. Therefore, the term proximal may refer to a direction that points into a patient's body, whereas distal may refer to a direction that points away from the patient's body.

The body member may be sized for placement substantially within the bladder and bladder neck, prostate urethra, with the distal terminating end located proximal to an external urethral sphincter to allow normal operation of the external sphincter. The coil retaining member may extend from the proximal end portion of the body member. The coil retaining member may be straightened into a first state to allow passage of the catheter into the urethra. The coil retaining member may be coiled into a second state when located in a bladder to hold the body member in place substantially within the urethra by removing a straightening stylet. The pusher segment may abut the distal tubular segment such that the device does not migrate as the straightening stylet is removed.

Embodiments of this aspect may include the following features. The coil catheter system may include a coil catheter, a connecting segment, a monofilament suture, and a pusher tubular device. The coil catheter may include a body member and a proximal coil retaining member. The coil catheter may include a swan neck portion, which is the transition from the tubular segment, and the coil may angle proximally then curve distally into a horizontal portion of the coil. The right angle and swan neck configuration may allow for retaining of the device as well as a tolerance to traction prior to removal. The length of the tubular segment, swan neck portion, and coil catheter may be changed to facilitate a variety of anatomical and gender-specific challenges. Multiple coils and a short tubular segment may be appropriate for females with short urethra which may allow for catheterization from inside the bladder outward and be controlled externally with a control suture. In patients with a dysfunctional sphincter, the device may require bridging the sphincter to empty the bladder then snapping back proximal to the sphincter to allow for continence. The invention described herein may be implemented as a dynamic catheterization device which has a passive state in the prostate fossa in men and bladder for females. For spinal cord patients, the invention described herein may be disposed by default at a location proximal to the urinary sphincter with dynamic positioning for bladder emptying while bridging the sphincter. Upon bladder drainage, the suture, without gently pulling, may revert back to a position proximal to the sphincter which allows for continence.

The coil may replace a balloon as a retaining member. The coil may include a tapered tip with a guide wire channel, with eyelets placed at various locations without limitation on the coil segment. The eyelet placed in the swan neck portion may be perpendicular to the direction of flow. The bladder mucosa trauma may be minimized due to such placement of the eyelets. The curve of the coil may be fashioned to have a swan neck portion which extends from the horizontal plane of the coil device, wherein the horizontal plane may be at a right angle to the tubular (e.g., stem) portion. The swan neck portion may allow for traction on the device prior to uncoiling of the horizontal member. While an inadvertent removal of an inflated balloon-type catheter may result in urethral mucosal injury, catheter mucosal injury may be minimized if inadvertent removal of the coil occurs, since the diameter of the device upon removal is the same as the diameter upon insertion.

The coil retaining member of the coil catheter may be a tube constructed with coil-shape memory. The coil retaining member may also be rounded at the ends of the tubing to provide user comfort during insertion of the catheter into the patient's urethra. A guide wire channel may allow for a guide wire to be utilized with difficult placement due to false passages in the urethra or other anatomical challenges.

The process for creating the swan neck, vertical, and dynamic portion of the device with a horizontal coil is a unique process encompassing features defined in the utility patent identified as Manufacturing Process for integration of retaining member with vertical component coupled with the horizontal coil. The vectors of pull may impact the device in two separate phases of uncoiling, and they may be coupled with a resistance against pull out which is initially absorbed by the vertical swan neck component.

Prior to and during insertion of the coil catheter into the patient's urethra, the coil retaining member may be in a straightened first state. The coil retaining member may return to substantially the second coil state once inside the patient's bladder and thereby may act as an anchor to keep the body member of the catheter substantially within the prostatic urethra.

In one embodiment, the body member may include one or more side openings to allow fluid to drain from the urethra. The lumen of the body member and length of the tube may be designed to be equivalent to a variety of tubular dimensions. One embodiment may include a tubular portion which is constructed to have a star shaped exterior which may allow urine to travel predominantly around rather than through the device.

In one embodiment, the suture may be long enough to extend from the body member to the outside of the patient's body. The suture may be constructed with monofilament nylon or other equivalent materials. The coil catheter may be removed easily from the patient's body by pulling downward on the suture. The end of the suture may be connected to a structure (e.g., snap cap, ball, ring, coil, or the like) that extends out of the body entirely. The purpose of the snap cap is to facilitate location of the suture's end and eventual removal of the coil catheter by simply pulling on the located suture, providing a gentle traction on the suture. In patients with diminished manual dexterity, a magnet may be placed on the snap cap and on the end of the stylet device. Such magnet may allow for engaging the snap cap with gentle traction and may be placed on the device to engage the device into the bladder in females or traverse the sphincter in male patients with sphincter resistance.

The device may utilize additional materials which may add qualities such as without limitation lubrication, hydrophilic coating, radiographic enhancing material, among others. The material for the device may include without limitation Carbothane™ and may be constructed with various inner diameters (ID), outer diameters (OD), stiffness, or the like. The pusher, stylet, and/or suture material may not be limited in size or characteristics.

In another embodiment, the invention relates to a coil catheter system for draining fluid from a patient's body cavity including without limitation bladder, stomach, colon, ileal loop, colostomy, and/or abdominal peritoneal cavity, among others.

In general, in still another aspect, the invention may allow for a variety of devices within the body cavity to be controlled and/or manipulated externally with a tethering suture. This may allow for episodic movement from a passive state of the device to an active state, which may change the fluid dynamics to favor voiding or continence. To void, the user may pull on the suture, causing the distal end of the coil to move into the bladder neck and through the sphincter valve, allowing the bladder to drain.

In another device iteration, the device may be fashioned to have an inverted umbrella membrane, which may occlude the bladder neck, allowing continence to be achieved.

Another iteration may include the capability of the coil being imbedded with various medications which may allow for a unique drug delivery into body cavities such as without limitation the bladder, stomach, colon, ileal loop, colostomy, and/or abdominal cavity, among others. The tethered control may also allow for manipulation of the device in the cavity. The device may be configured with monitoring devices allowing for wireless transmission of images or data.

In another embodiment, the catheter may be used for cervical dilation to induce labor.

In yet another embodiment, the catheter may be used for widening the nasal passages to treat sleep disorders (e.g., sleep apnea).

The coil catheter may allow the bladder to fill and contract in synchronous sphincter relaxation without prostate urethral resistance. This feature may allow for defining the functional capacity of both the bladder and the urinary sphincter. In patients with chronic overdistension due to prostate obstruction, the device may act as a bladder rehabilitation device as well as a bladder neck, and/or provide prostate fossa dilation. With acute urinary or chronic retention, the improvement in bladder muscular contraction in a volitional manner may obviate the need for a variety of interventional procedures, which may focus on only the reduction in prostate resistance but may not have the capacity to enhance the vesicular (bladder) pressure; an enhanced vesicular pressure, when coupled with a reduction in the prostate resistance, may increase the flow rate of fluids in patients. More efficient voiding, with reduced residual urine in the bladder, coupled with a competent urinary sphincter and elimination of the need for an external collection device, may result in a collage of clinical improvements.

The coiled shape has been utilized in a variety of medical devices to resist removing of a straight tubular device. The unique feature of the current vertical and horizontal design includes the horizontal portion disposed distally with respect to the apex of the device. Proximal refers to an inward direction toward the patient, whereas distal refers to an outward direction away from the patient. Prior coiled shapes may include an extension proximal to the tubular device. With tension on these designs, uncoiling may begin with any tension on the distal tubular portion. The current design may protect the uncoiling of the horizontal component from the initial tension. The swan neck portion may allow for lengthening of the vertical tubular component and, upon lifting the tension, the vertical tubular portion may “snap back” to its original configuration due to a horizontal stabilizing effect. The distance for the extension and snap back may allow for a variety of medical applications with an innate resistance against inappropriate migration, which is common in “pig tail curl” or “J” shaped tips. The application defines the unique manufacturing process to construct the vertical—swan neck—horizontal-shaped device.

These and other aspects and features of nonlimiting embodiments of the present invention will become apparent to those skilled in the art upon review of the following description of specific nonlimiting embodiments of the invention in conjunction with the accompanying drawings.

### BRIEF DESCRIPTION OF THE DRAWINGS

For the purpose of illustrating the invention, the drawings show aspects of one or more embodiments of the invention. The drawings constitute a part of this specification and include exemplary embodiments of the present invention illustrating various objects and features thereof. However, it should be understood that the present invention is not limited to the precise arrangements and instrumentalities shown in the drawings, wherein:

FIG. 1 is a diagrammatic representation of a preferred embodiment of the present invention shown in a typical environment;

FIG. 2 is a front elevational view of a preferred embodiment of the present invention shown in a straightened orientation;

FIG. 3 is a top plan view of the embodiment shown in FIG. 2;

FIG. 4 is a bottom plan view of the embodiment shown in FIG. 2;

FIG. 5 is a top plan view of a preferred embodiment of the present invention in a coiled orientation;

FIG. 6 is a front elevational view of the embodiment shown in FIG. 5;

FIG. 7 is a three-dimensional isometric view of the embodiment shown in FIG. 5;

FIG. 8 is a front elevational view of a preferred embodiment of the present invention in combination with a typical pusher device;

FIG. 8A is a detailed view of a portion thereof taken about the circle 8A in FIG. 8;

FIG. 9 is a diagrammatic representation of an embodiment of the present inventions shown in a typical environment;

FIG. 9A is a detailed view of a portion thereof taken about the circle 9A in FIG. 9;

FIG. 10 is a diagrammatic representation of an embodiment of the present invention shown without an external container;

FIG. 10A is a detailed view of a portion thereof taken about the circle 10A in FIG. 10;

FIG. 10B is a detailed view of a portion thereof taken about the circle 10B in FIG. 10;

FIG. 11 is a three-dimensional view showing the preferred embodiment of the present invention in combination with a pusher;

FIG. 11A is a detailed view of a portion thereof taken about the circle 11A in FIG. 11;

FIG. 12 is a three-dimensional view showing a first manufacturing step for manufacturing a preferred embodiment of the present invention;

FIG. 13 is a three-dimensional isometric view showing a second manufacturing step thereof;

FIG. 14 is a three-dimensional isometric view showing a third manufacturing step thereof;

FIG. 15 is a three-dimensional isometric view showing a fourth manufacturing step thereof;

FIG. 16 is a three-dimensional isometric view showing a fifth manufacturing step thereof;

FIG. 17 is a three-dimensional isometric view showing a sixth manufacturing step thereof;

FIG. 18 is a three-dimensional isometric view showing a seventh manufacturing step thereof;

FIG. 19 is a flow chart diagramming a method of using a preferred embodiment of the present invention; and

FIG. 20 is a flow chart diagramming a method of manufacturing a preferred embodiment of the present invention.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

### I. Introduction and Environment

As required, detailed aspects of the present invention are disclosed herein. However, it is to be understood that the disclosed aspects are merely exemplary of the invention, which may be embodied in various forms. Therefore, specific structural and functional details disclosed herein are not to be interpreted as limiting, but merely as a basis for the claims and as a representative basis for teaching one skilled in the art how to variously employ the present invention in virtually any appropriately detailed structure.

Certain terminology will be used in the following description for convenience in reference only and will not be limiting. For example, up, down, front, back, right and left refer to the invention as orientated in the view being referred to. The words, “inwardly” and “outwardly” refer to directions toward and away from, respectively, the geometric center of the aspect being described and designated parts thereof. Forwardly and rearwardly are generally in reference to the direction of travel, if appropriate. Said terminology may include the words specifically mentioned, derivatives thereof and words of similar meaning.

The directional terms proximal and distal may require a point of reference. In this application, the point of reference in determining direction may be from the perspective of a patient. Therefore, the term proximal may refer to a direction that points into a patient's body, whereas distal may refer to a direction that points away from the patient's body.

### II. Preferred Embodiment Catheter System 2

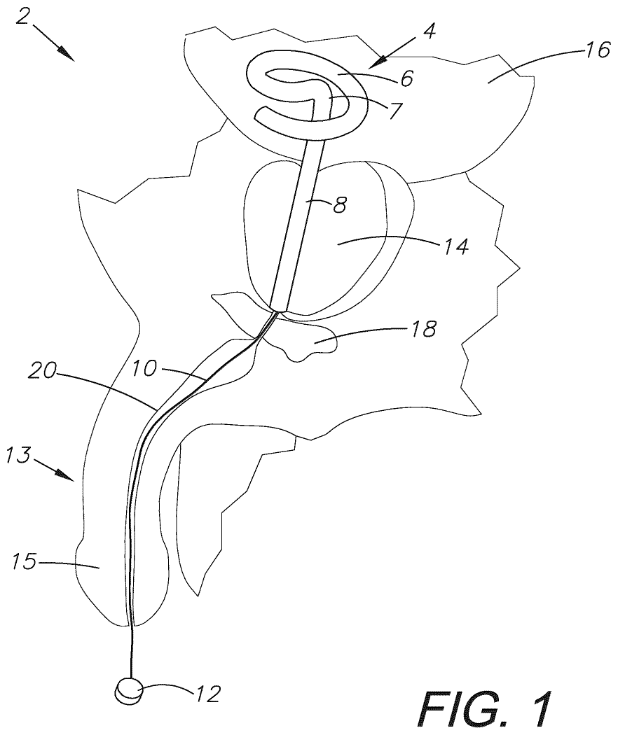

As shown in a typical environment in FIG. 1, the present invention is a catheter system 2 which features a coiled catheter 4 having a halo portion 6 connected to a stem portion 8 via a swan neck portion 7, such that the halo portion 6 is disposed along a plane perpendicular to the direction of the stem portion 8. Halo portion 6 may be located within a bladder 16 once properly placed and may serve to provide optimal flow out of catheter 4 through stem portion 8. FIG. 1 shows a thread 10 connected to a snap cap 12 located outside of the body to prevent the thread 10 from being drawn up into the body, and instead the thread 10 would be stopped at the glans 15 of a penis 13 (as shown). This may function similarly in a female patient. Thread 10 may be formed from a monofilament suture-type material in a preferred embodiment. Snap cap 12 may include a magnet or be made of a magnetic material.

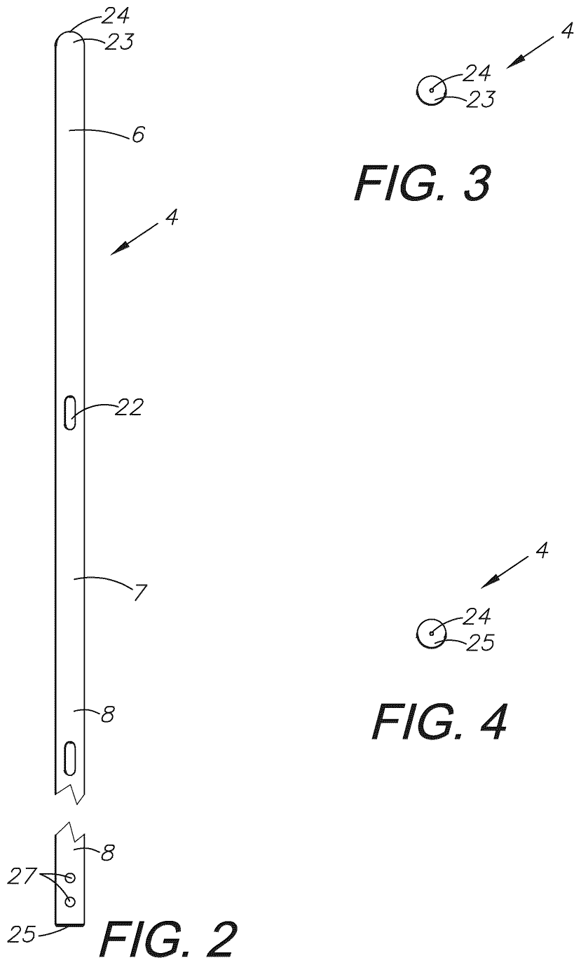

Stem portion 8 of catheter 4 may pass through a prostate gland 14 and the end of the stem portion 8 may be located in proximity to an external sphincter 18. This system may facilitate flow from bladder 16 through catheter 4, via eyelets 22, which may be located at swan neck portion 7 and/or at a proximal coil tip 23 of halo portion 6, and out through the urethra 20 (see FIG. 2). It is important that stem portion 8 functions as a short straight arm that un-obstructs the prostatic urethra and sits above the urinary sphincter 18. In other words, the device does not retain the urinary sphincter in an open orientation. This configuration may allow for volitional voiding of a bladder by a patient, facilitated by catheter 4, without incontinence.

As shown in FIG. 1, when catheter 4 is inserted into the bladder, halo portion 6 may coil, forming swan neck portion 7 which terminates into a right-angle bend; these elements may together form a stabilizing elbow which ensures that catheter 4 remains properly in place within a bladder for optimal drainage through the catheter 4.

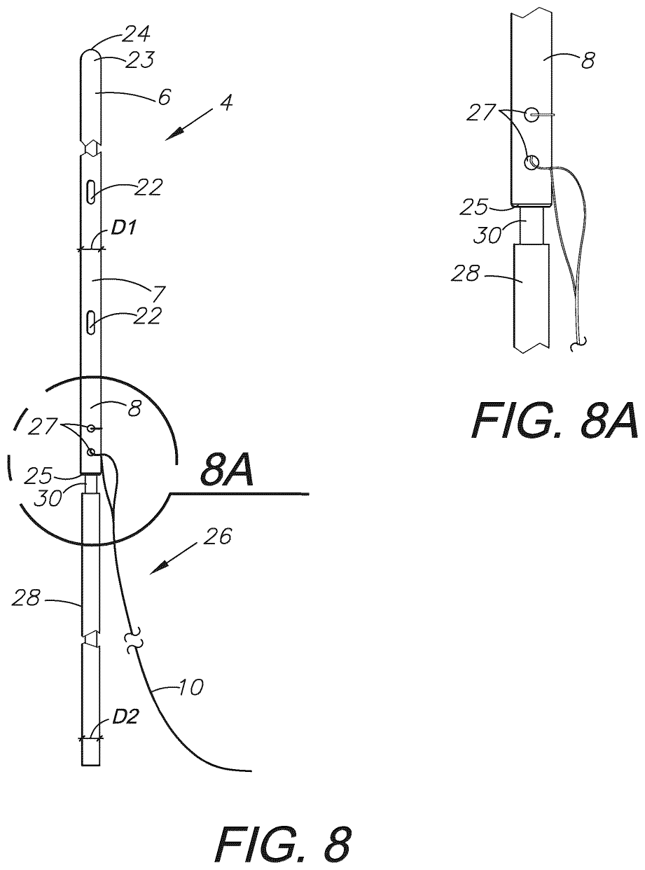

FIGS. 2-4 show how the catheter 4 may be inserted into a body in a straight orientation for easy placement. A guidewire hole 24 may be located at proximal coil tip 23 of halo portion 6 and at a base 25 of stem portion 8 for use with a pusher 26 and a stylet, as is typical and as is shown in more detail in FIG. 8. As shown in FIG. 2, proximal coil tip 23 may be tapered for easy insertion.

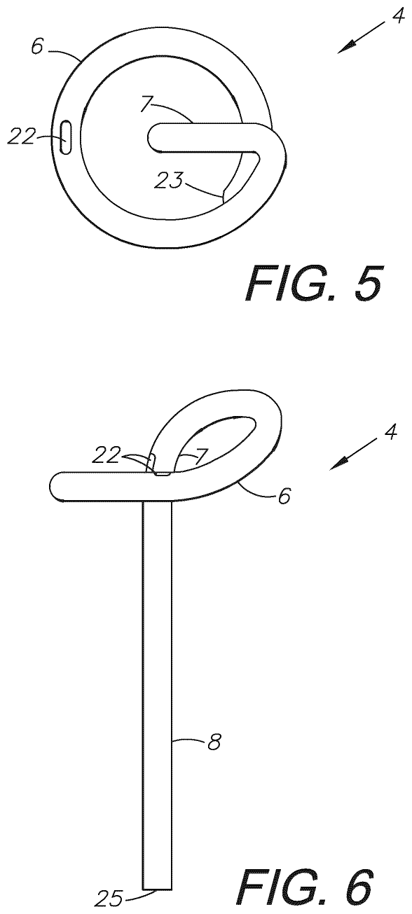

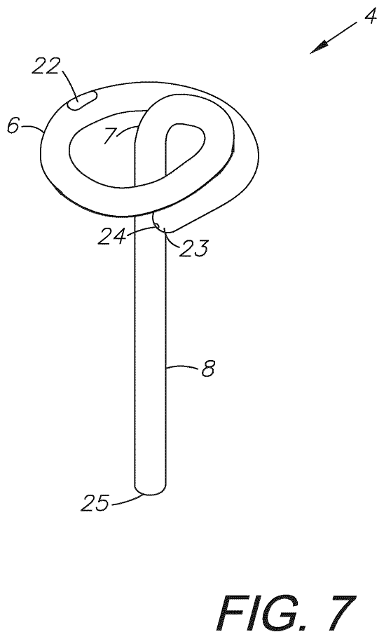

FIGS. 5-7 show how catheter 4 may adopt a coiled configuration once inserted into its proper environment in bladder 16, with halo portion 6 and stem portion 8 connected through swan neck portion 7. Catheter 4 may default to this form due to a manufacturing process discussed below. Once the guidewire of stylet and pusher 26 is removed (see FIG. 8) or at least withdrawn slightly, catheter 4 may automatically coil into the form shown.

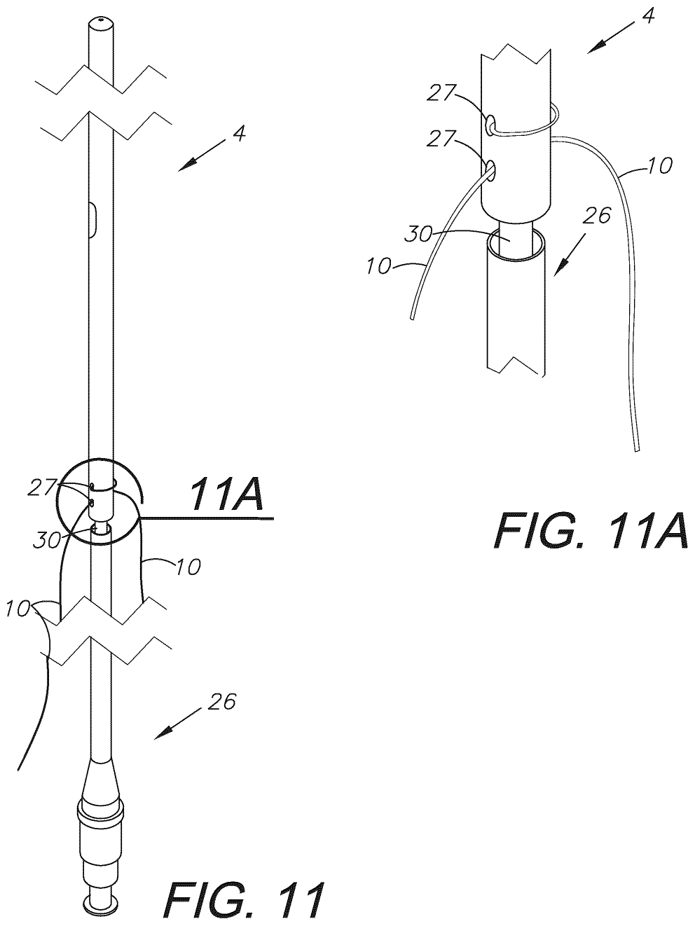

FIGS. 8 and 8A show catheter 4 in its straight orientation in combination with a pusher 26 having an outer tube 28 and a stylet 30. Thread 10 and snap cap 12 may extend through pusher 26 and connect to the end of catheter 4 near its base 25 or may be located externally to the pusher 26 and threaded through receivers 27 of stem portion 8 of the catheter 4, as shown in more detail in FIG. 11. FIGS. 8 and 11 also show how pusher 26 may have a smaller diameter than that of catheter 4, further easing insertion of the catheter.

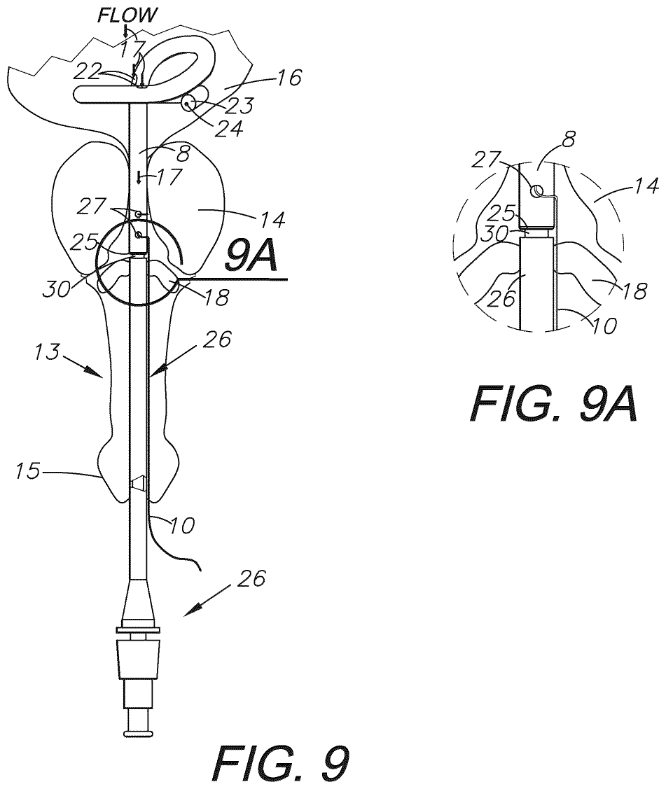

FIG. 9 shows catheter 4 and pusher 26 in the typical environment shown in FIG. 1, where the catheter 4 is fully inserted and coiled in position. Flow 17 is indicated via the arrows through catheter 4 by way of eyelets 22, vertically downward rather than perpendicular to catheter 4 as is the case with prior art catheters. The detailed view of FIG. 9A shows how thread 10 may be retained through receivers 27 in stem portion 8 to secure the thread 10 to catheter 4 for removal. Thread 10 is shown to be external to pusher 26, which abuts base 25 of catheter 4, with stylet 30 contacting the base 25 of the stem portion 8 thereof.

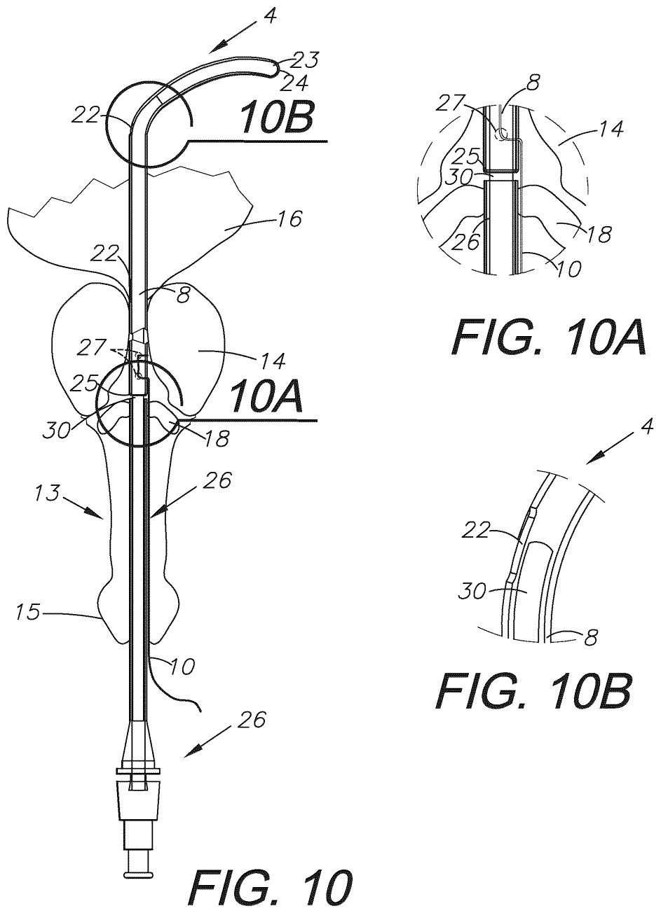

FIG. 10 shows the insertion of catheter 4 using pusher 26 in a sectional view so as to better show the internal components thereof. FIGS. 10A and 10B show additional detail about their respective circles in FIG. 10, such as FIG. 10B showing how stylet 30 of pusher 26 may be implemented using a functional guidewire that can be placed up and into catheter 4 itself to help position the catheter 4 within a bladder. Catheter 4 may be straightened by internally placed stylet 30 with guidewire. Catheter 4 as shown may be placed over guidewire for safe insertion into the bladder, after which the guidewire and stylet 30 may be removed, leaving only the coiled and unencumbered catheter 4 in a bladder, as shown in FIG. 1.

FIG. 11 and FIG. 11A show in more detail the extra-luminal suture thread 10 which does not obstruct the lumen of catheter 4. Similar to the positioning of stem portion 8 above sphincter 18, this configuration is intended to prevent incontinence and may be used to safely remove catheter 4 at a later date. No sheath may be required for such procedure.

### III. System 52 for Manufacture of Catheter System 2

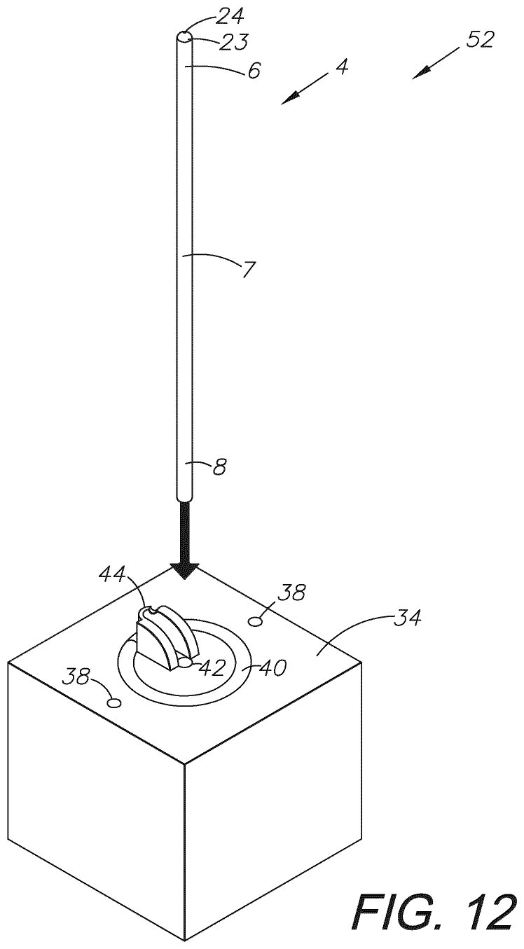

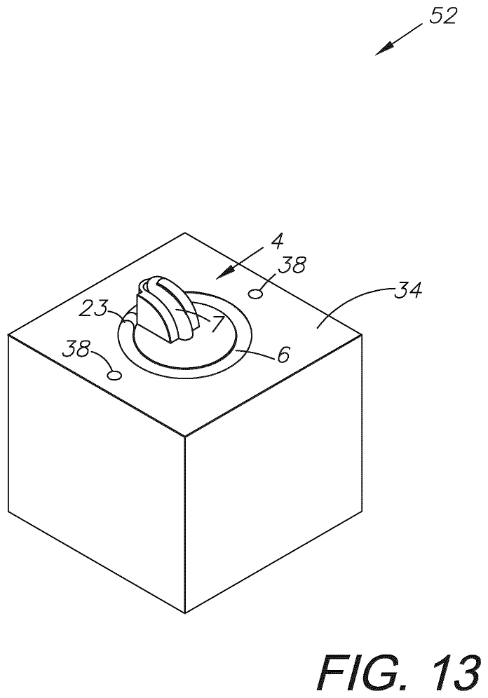

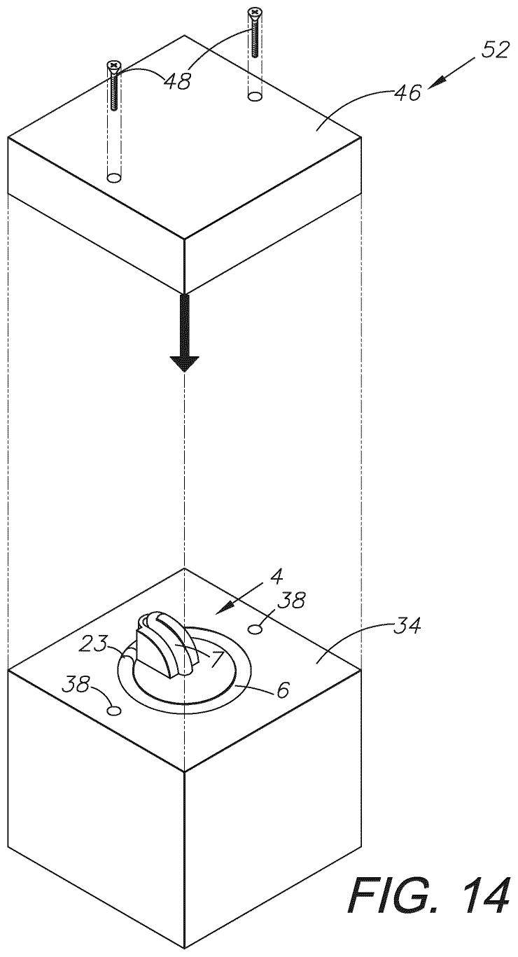

FIGS. 12-18 show a manufacturing system 52 for manufacturing coil catheter 4. As shown, coil catheter 4 may be formed from an originally straight catheter tube by placing it into a mold base 34. A flexible yet solid tubing support may be inserted into the catheter tube prior to molding to prevent kinks during the forming process. Mold base 34 may include receiver slots 38 for screws 48 to receive a mold cap 46, as shown in FIG. 14. Mold base 34 may also have a forming block with a first form, such as a swan neck form 44, and a second form, such as a halo form 40. Stem portion 8 may be inserted into a stem receiver 42 and swan neck portion 7 may be placed into swan neck form 44, and halo portion 6 may be curled around in halo form 40. Mold cap 46 may then be secured to mold base 34 via screws 48.

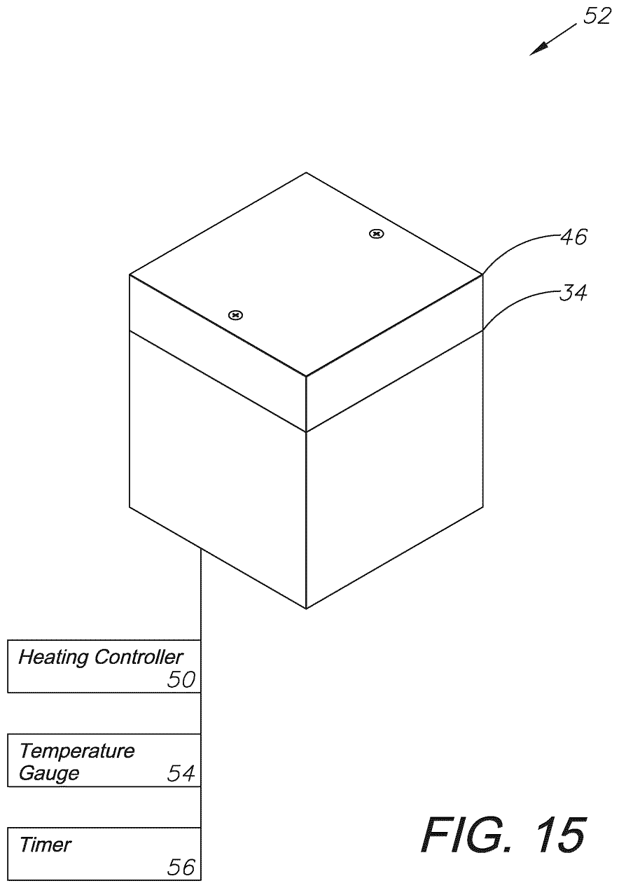

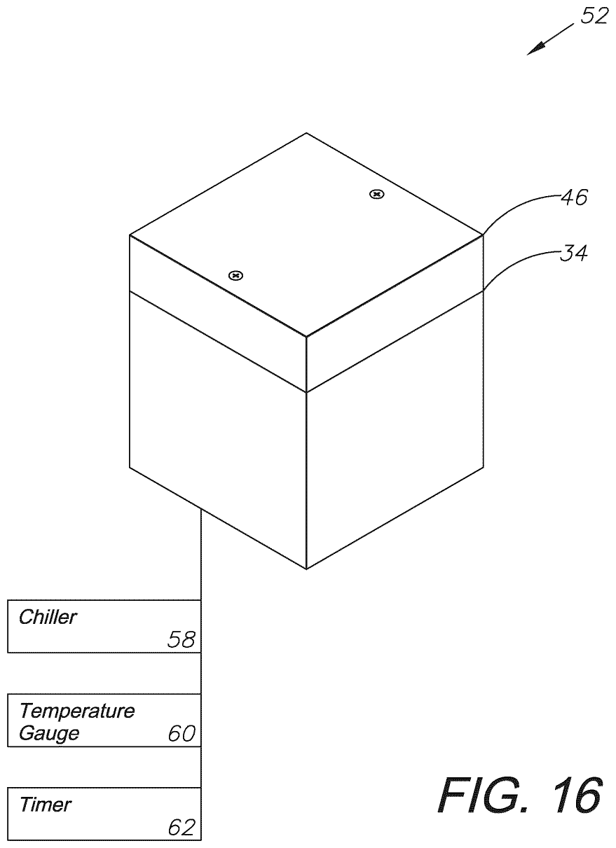

FIG. 15 shows a heating controller 50 with a temperature gauge 54 and a timer 56. Mold base 34 may be heated to an appropriate temperature to thermoset coil catheter 4. This process may take approximately 15-19 minutes to reach the proper temperature, at which mold base 34 may be held for a period of time, such as 15 minutes. FIG. 16 shows a chiller 58 with a temperature gauge 60 and a timer 62. Chiller 58 may set a temperature of 5.00 degrees Celsius and cool a heated coil catheter 4 down to thermoset its shape. A temperature alarm may be included to properly track chilling. Once temperature gauge 60 indicates a temperature less than 80 degrees Fahrenheit, typically after 15-19 minutes, chiller 58 may be turned off.

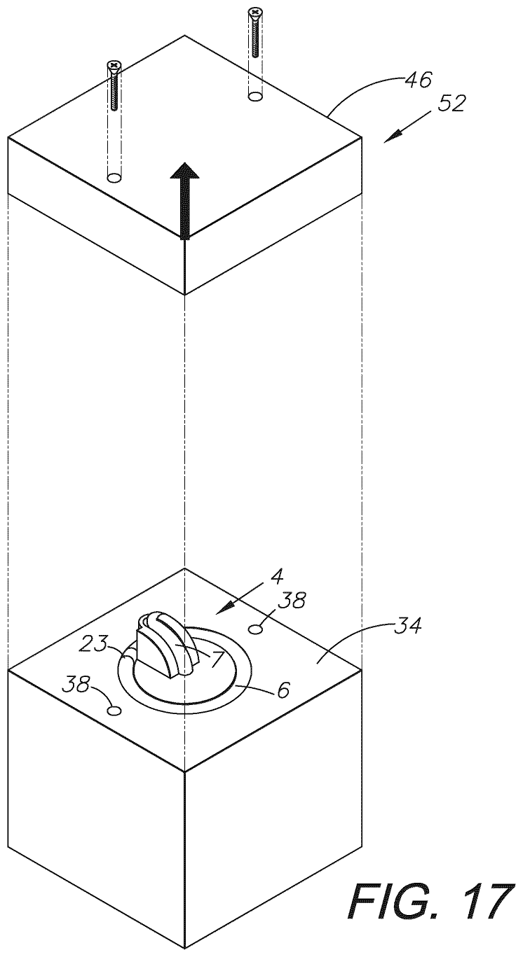

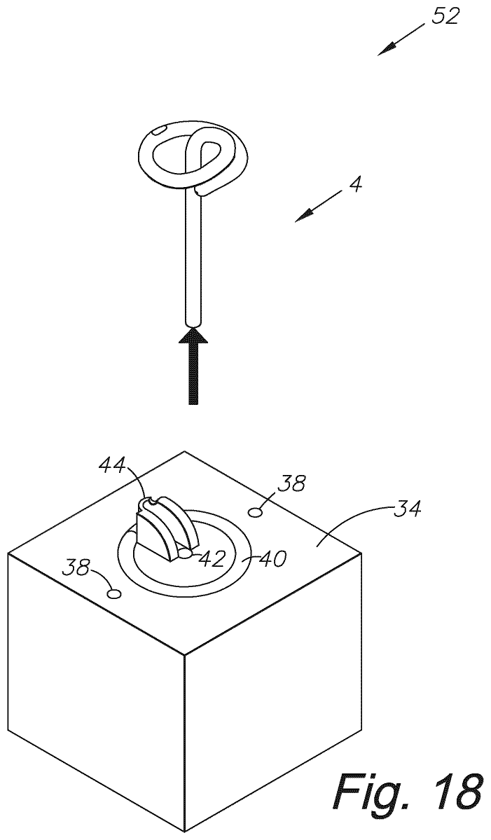

Mold cap 46 may be removed, as shown in FIG. 17, and a thermoset coil catheter 4 may be removed from mold base 34, as shown in FIG. 18. After this step, coil catheter 4 may be placed on a rack for further cooling and should be covered to reduce contamination risks.

### IV. Method 102 of Using Catheter System 2

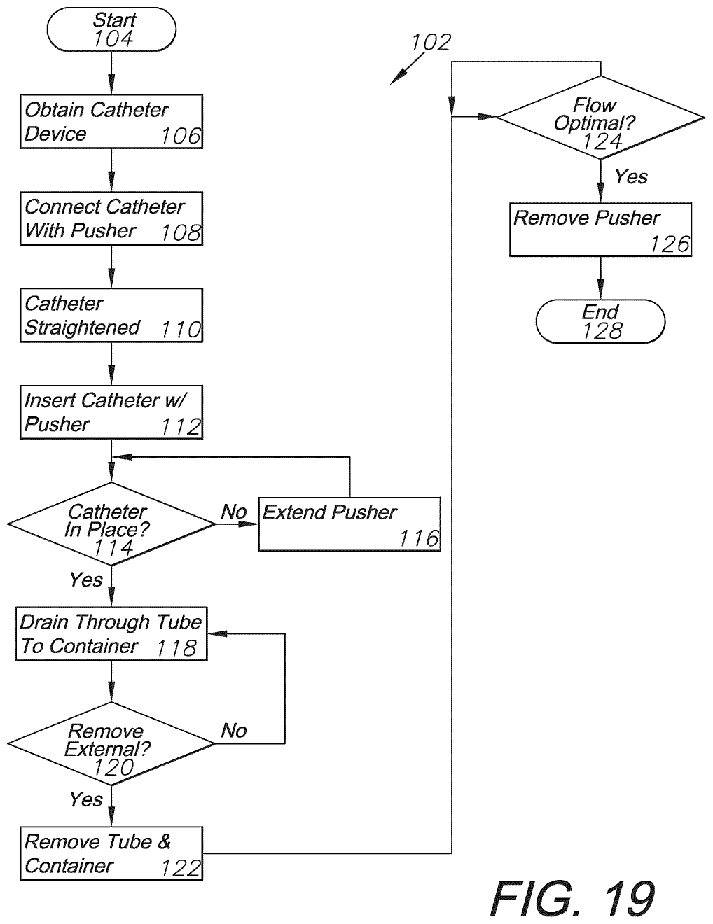

FIG. 19 shows the steps taken in practicing a method 102 of using coil catheter system 2 as described above. Method 102 starts at step 104, where catheter 4 is obtained at step 106. A pusher 26 is obtained and used with catheter 4 at step 108, and the catheter 4 is straightened as shown in FIG. 8 at step 110. This allows the catheter 4 to be inserted into a body at step 112 using pusher 26. A check of whether the catheter is in place at step 114 may require pusher 26 to be extended at 116 to ensure proper placement of the catheter. Once in place at step 114, the bladder may drain through eyelet 22 into the catheter 4, out through pusher 26, through receiver 27, and into an external container (not shown) at step 118. A determination is made at step 120 regarding whether to remove the tube and external container. If not, they may remain in place. If so, then the tube and container may be removed at step 122.

A check is then made at step 124 and a determination may be made regarding whether flow is optimal with pusher 26 in place. If not, pusher 26 may remain in place. If so, pusher 26 may be removed at step 126 so that flow may be entirely facilitated using catheter 4 in the body. The process may then end at step 128 until such a time that pusher 26 is to be removed.

### V. Method 152 of Manufacturing System 52 for Manufacture of Catheter System 2

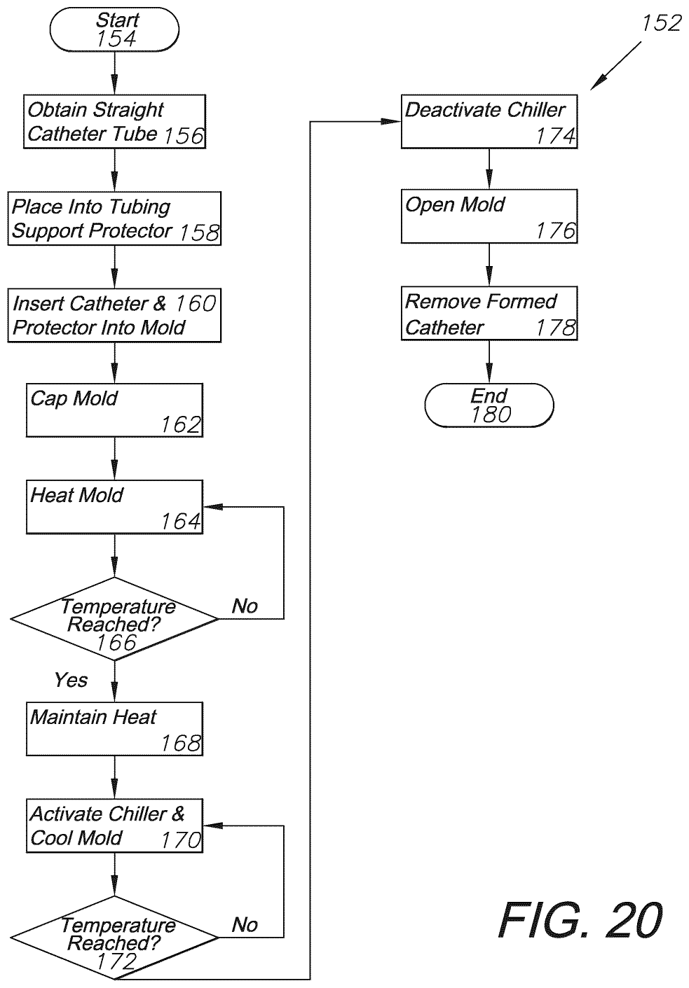

FIG. 20 shows the steps taken in practicing a method 152 of manufacturing coil catheter 4, as described above using manufacturing system 52. Method 152 starts at 154, where a straight catheter tube may be obtained at step 156. This catheter tube should be cut to size, approximately 8.5″ long, and may be outfitted with a tubing support protector to reduce the risk of tubing kinks during the forming process at step 158.

The catheter and protector may then be inserted into mold base 34 at step 160, using swan neck form 44 and halo form 40 of the mold base 34. Mold base may be capped at step 162 and heated at step 164, as described above. A check using temperature gauge 54 may be made at step 166 to determine if a proper temperature has been reached. If not, heating may continue. If so, this proper temperature may be maintained at step 168 for 15 minutes, after which chiller 58 may be activated at step 170 to cool the mold base 34.

A check may be made to determine if the mold has reached a temperature threshold, e.g., a cooled temperature below 80 degrees Fahrenheit, at step 172. If not, chilling may continue. If so, then chiller 58 may be deactivated at step 174, mold cap 46 may be opened at step 176, and a formed catheter 4 may be removed at step 178, ending the process at step 180.

The catheter may be manufactured using Carbothane™, a registered trademark owned by Lubrizol Advanced Materials, Inc. of Cleveland, OH for a family of medical-grade polycarbonate-based aliphatic and aromatic polyurethanes, or other materials which may provide long, safe use and biocompatibility.

The foregoing has been a detailed description of illustrative embodiments of the invention. It is to be understood that while certain embodiments and/or aspects of the invention have been shown and described, the invention is not limited thereto and encompasses various other embodiments and aspects. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A method of manufacturing a catheter, the method comprising:
placing a catheter tube into a mold base, wherein the mold base comprises a forming block having a first form and a second form;
positioning a first portion of the catheter tube within the forming block;
capping the mold base with a mold cap;
heating the mold base with a heating element;
cooling the mold base with a chiller, wherein:
the chiller is connected to a temperature gauge; and
cooling the mold base comprises cooling the mold base as a function of the temperature gauge;
deactivating the chiller as a function of the temperature gauge;
removing the mold cap from the mold base; and
removing a thermoset catheter tube from the mold base, wherein
the thermoset catheter tube comprises a proximal end and a distal end; and
the proximal end comprises a coil having a first portion and a second portion.

2. The method of claim 1, wherein:
the first form comprises a swan neck form; and
the second form comprises a halo form.

3. The method of claim 1, wherein:
the mold base further comprises a stem receiver; and
placing the catheter tube into the mold base comprises placing a second portion of the catheter tube into the stem receiver.

4. The method of claim 1, wherein the distal end comprises a tubular portion.

5. The method of claim 4, wherein the second portion is disposed in a horizontal plane that is at an angle with the tubular portion.

6. The method of claim 5, wherein the angle comprises a right angle.

7. The method of claim 4, wherein the tubular portion comprises a star-shaped exterior, thereby allowing liquid to travel around rather than through the catheter.

8. The method of claim 1, wherein the proximal end further comprises a tapered end.

9. The method of claim 1, wherein the thermoset catheter tube comprises at least an eyelet, wherein the at least an eyelet is disposed in a direction perpendicular to a direction of flow and configured to facilitate and enhance flow through the catheter.

10. The method of claim 1, wherein the thermoset catheter tube is configured to move between a coiled state and an uncoiled state.

11. The method of claim 1, wherein:
the first portion comprises swan neck portion; and
the second portion comprises a halo portion.

12. The method of claim 1, wherein:
the heating element is connected to the temperature gauge; and
heating the mold base comprises heating the mold base as a function of the temperature gauge.

13. The method of claim 12, wherein:
the temperature gauge is configured to identify at least a first temperature threshold; and
heating the mold base comprises deactivating the heating element as a function of an indicated temperature outside of the at least a first temperature threshold.

14. The method of claim 1, wherein the temperature gauge is configured to identify at least a second temperature threshold.

15. The method of claim 14, wherein deactivating the chiller comprises deactivating the chiller as a function of an indicated temperature outside of the at least a second temperature threshold.

16. The method of claim 14, wherein at least a second temperature threshold includes 5 degrees Celsius.

17. The method of claim 1, wherein the temperature gauge comprises a temperature alarm configured to track a heating or chilling process.

18. The method of claim 1, further comprising:
placing a tubing support protector into the catheter tube prior to placing the catheter tube into the mold base.

19. The method of claim 1, further comprising:
forming a stabilizing elbow using the first portion and the second portion, wherein the stabilizing elbow terminates into a straight stem at the distal end of the thermoset catheter tube.

20. The method of claim 1, further comprising:
providing a portion configured to place a suture external to a lumen of the thermoset catheter tube and external to a pusher, thereby being configured to prevent obstruction and tethering of the catheter and to allow for bridging a sphincter, positioning the catheter, and removing the catheter.
